(19) United States
(12) Patent Application Publication
(10) Pub. No.: US 2016/0015860 A1
(43) Pub. Date: Jan. 21, 2016

(54) MICRO-TISSUE PARTICLES AND METHODS FOR THEIR USE IN CELL THERAPY

(71) Applicant: UNIVERSITY OF WASHINGTON THROUGH ITS CENTER FOR COMMERCIALIZATION, Seattle, WA (US)

(72) Inventors: Charles Murry, Seattle, WA (US); Kareen Louise Kreutziger, Seattle, WA (US)

(21) Appl. No.: 14/379,294

(22) PCT Filed: Feb. 15, 2013
(86) PCT No.: PCT/US13/26512

§ 371 Date: Aug. 16, 2014

Related U.S. Application Data
(60) Provisional application No. 61/599,867, filed on Feb. 16, 2012.

(51) Int. Cl. A61L 27/38; A61L 27/54

(52) U.S. Cl. CPC A61L 27/3826; A61L 27/3886; A61L 27/3808; A61L 27/3873; A61L 27/3834; A61L 27/54; A61L 2300/64; A61L 2300/25; A61L 2300/426; A61L 2400/06; A61L 2430/20

(57) ABSTRACT

In some embodiments, a micro-tissue particle comprising a scaffold-free population of aggregated cells is provided. The micro-tissue particle may have a diameter less than approximately 1 mm. In some aspects the diameter is less than approximately 500? m. The population of cells may include at least one terminally differentiated cell type. In one aspect, the population of cells may include cardiomyocytes, endothelial cells, smooth muscle cells, mesenchymal stem cells, or a combination thereof. The micro-tissue particle may be used to treat or regenerate an injured, degenerated or diseased tissue. For example, micro-tissue particles that include cardiomyocytes may be administered to myocardial tissue that has been injured due to a myocardial infarction.

### PRIORITY CLAIM

This application claims priority to U.S. Provisional Patent Application No. 61/599,867 filed Feb. 16, 2012, the subject matter of which is hereby incorporated by reference as if fully set forth herein.

### STATEMENT OF GOVERNMENT INTEREST

The present invention was made with government support under Grant No NIH R01 HL084642, awarded by the National Institutes of Health. The Government has certain rights in the invention.

### BACKGROUND

Stem cell-based therapy and transplantation using engineered cells and tissues is thought to hold great potential for restoring function to a variety of injured, depleted or degenerated tissues, including the myocardium, bone, blood and marrow, spinal cord and other motor nerves, and brain nuclei.

Cell preparations that are currently being used in clinical trials to investigate the potential of stem cell therapy utilize single cell suspensions. Pre-clinical use of engineered macro-sized tissue referred to herein as “tissue patches” will likely provide additional advances for stem cell therapy. Clinical success of stem cell therapy using current single cell preparations has been limited due to several factors including cell death and low engraftment efficiency. Further, although promising, tissue patches will likely require a surgical or other invasive method of transplantation. Therefore, there is a need in the art for a cell preparation that is minimally invasive and will remain viable once transplanted, and can fully integrate into a host tissue without adverse reaction for uses of treating or regenerating an injured tissue.

### SUMMARY

In one embodiment, a micro-tissue particle comprising a scaffold-free population of aggregated cells is provided. The micro-tissue particle may have a diameter less than approximately 1 mm. In some aspects the diameter is less than approximately 500 μm, The population of cells may include at least one terminally differentiated cell type selected from cardiomyocytes, endothelial cells, smooth muscle cells, pancreatic α-cells, pancreatic β-cells, pancreatic δ-cells, pancreatic γ-cells, osteoblasts, osteoclasts, osteocytes, chondrocytes, epithelial cells, keratinocytes, melanocytes, myocytes, fibroblasts, oligodendrocytes, motor neurons, RPE cells, dopaminergic neurons, hepatocytes, dermal papilla cells, thecal cells, follicular cells, luteal cells, leydig cells, sertoli cells glomerular parietal cells, podocytes, proximal tubule brush border cells, parenchymal cells, marrow stromal cells, fibroblasts, plasma cells, neutrophils, monocytes, myeloid cells, endothelial cells, gut epithelial cells, parietal cells, gut endocrine cells, or a combination thereof.

In another embodiment, a pharmaceutical composition that includes a micro-tissue particle is provided. In addition to the micro-tissue particle, the pharmaceutical composition may include a carrier, one or more graft enhancement agent, or a combination thereof. In some aspects, the graft-enhancement agent may include immunosuppressive agents (e.g., cyclosporine A), antibiotics, extracellular matrix elements, anti-apoptotic agents, anti-ischemic agents, anti-toxicity agents, anti-apoptotic agents, pro-survival agents, pro-proliferation agents, or a combination thereof.

In another embodiment, a method for treating an acute or pathologically injured target tissue is provided. Such a method may include a step of administering a therapeutically effective amount of a pharmaceutical composition, the pharmaceutical composition comprising a micro-tissue particle. In one embodiment, the pharmaceutical composition is administered by injection. The method may be used to treat any acute or pathologically injured target tissue, such as a myocardial tissue, a blood vessel, a pancreatic islet, a bone, cartilage, a skeletal muscle, a tendon, a ligament, an epidermis, a spinal cord, an eye, a nervous tissue, a liver, a hair follicle, an ovary, a testis, a kidney, bone marrow, an intestine, or a stomach.

### BRIEF DESCRIPTION OF THE DRAWINGS

FIG. 1 illustrates formation of micro-tissue particles (MTPs) under varying conditions. A, MTPs formed with only HUVECs in a 96-well round-bottom plate show graded diameter based on the input number of cells. Diameters are shown; all values are significantly different (P<0.001). Pictures (top) of live-cell MTPs correspond to abscissa. Scale bar: 300 μm. B, MTPs with 4000 cells per MTP form overnight as hanging drops after heat shock on the day prior (−1) or day of (0) MTP culture with variation in diameter. HUVEC:hMSC is a 2:1 ratio. *P<0.01 vs. day −1 heat shock. C, Human CD31 stain (DAB, brown) marks HUVECs in “vascular” MTPs (HUVEC:hMSC is 2:1). 2000 cells per MTP were formed in hanging drops. Scale bar: 100 μm; sectioning plane through MTPs varies. E, Alpha smooth muscle actin (red) marks human aortic smooth muscle cells in MTPs formed from approximately 50 cells each in microwells. Scale bar: 25 μm.

FIG. 2 shows that purity of cardiac MTPs depends on culture time and medium conditions. A, Cardiac MTPs enrich for cardiomyocytes (labeled by βMHC) over 5 days of culture in RPMI-B27 medium in microwells. *P<0.01 vs. day 1. Example images of cardiac MTPs shows increased βMHC staining (brown) at day 4 vs. day 1. Scale bar: 100 μm. B, Cardiac purity at day 4 varies with culture medium as shown by example images of cardiac MTPs in huEB (left), RPMIB27 with 0.125% methyl cellulose (middle), and RPMI-B27 with 20% FBS (right). *P<0.01 vs. huEB.

FIG. 3 illustrates engraftment of MTPs in the rat heart. A, Tri-cell “myocardial” MTPs of IMR90 hiPSC-derived cardiomyocytes, HUVECs and hMSCs (2:2:1 cell ratio) engraft in the border zone of an infarcted heart in an athymic rat. Collagenous scar is shown by picrosirius red with fast green counterstain for cytoplasm. B, Human grafts shown by a human pancentromeric probe (brown nuclei, top) are surrounded by cardiac troponin T-positive tissue of the rat host (pink) and show human lumens forming after one week by hCD31 stain (brown, bottom). Scale bar: 0.5 mm. C, βMHC-positive hESC-derived cardiomyocytes (brown) demonstrate engraftment of cardiac micro-tissue particles in an uninjured rat heart (top; scale bar: 0.5 mm) and show striations (arrow heads, bottom; scale bar: 50 μm).

FIG. 4 demonstrates that MTP engraftment has improved electrical coupling to the host at 4 weeks versus cell injections with comparable graft size and heart function. A, Cardiac MTPs formed intramyocardial grafts, double labeled with GFP (green, to label implanted cells) and α-actinin (red, to label cardiomyocytes; left) that were largely cardiomyocytes (middle; input was >50% cardiac) and showed striations at high magnification (right). Scale bar: 200 μm (left), 25 μm (right). B, Histological assessment of graft size at 2 and 4 weeks (as GFP+ percent of left ventricular (LV) area) shows that graft size at either 2 or 4 weeks is not different between MTPs and single cardiomyocyte cell injections (“Cells”). C, Echocardiography shows significant decline in heart function as measured by fractional shortening (FS) after the induction of a myocardial infarction (Baseline measurement). Treatment with MTPs or cells prevented further decline of FS but showed no difference at 2 and 4 weeks between groups. D, Coupling between host and graft was assessed ex vivo with fluorescence imaging of the graft using GCaMP3-positive cardiomyocytes (which causes a green flash when intracellular calcium increases with each beat). Image shows the graft region of interest (red box). Correlation of graft electrical activity with the host electrocardiogram (ECG, red trace) showed coupling (dotted lines) during spontaneous sinus rhythm (blue fluorescence trace) that was maintained during external stimulation up to 6 Hz (green trace) for MTP implants only. Summary table shows that MTP grafts were superior to single cell injections in their ability to couple to the host and be paced.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Abbreviations: βMHC, beta-myosin heavy chain; hCD31, human CD31; hESC, human embryonic stem cell; hiPSC, human induced pluripotent stem cell; hMSC, human mesenchymal stem cell or human marrow stromal cell; huEB, human embryiod body culture medium; HUVEC, human umbilical vein endothelial cell; IMR90, the name of the human fibroblast cell line used to generate hiPSCs by WiCell Research Institute; LVEDD, left ventricular end diastolic dimension; LVESD, left ventricular end systolic dimension; MEF, mouse embryonic fibroblast; MTP, micro-tissue particle.

Micro-Tissue Particles and their Preparation

Micro-tissue particles, methods for preparing micro-tissue particles, and therapeutic uses thereof are provided herein. The micro-tissue particles described in this disclosure may be used as a cell-based therapy for treating an injured, depleted or degenerated tissue in regenerative medical treatment methods as described below.

In some embodiments, a micro-tissue particle (MTP) includes a scaffold-free population of aggregated cells. A “scaffold-free” population of cells, as referred to herein, is an assembly or aggregate of two or more cells and the matrix components that the cells secrete. A scaffold-free population of cells does not include a synthetic or bioengineered matrix scaffold or gel that is commonly used in in vitro tissue engineering techniques to generate tissue patches or grafts. The use of a scaffold-free MTP is advantageous in a cell-based therapy such as those described herein because omitting a synthetic or bioengineered matrix scaffold diminishes the host immune response to the implant, thereby eliminating or reducing a host's unfavorable immune response to such exogenous biomaterials.

In some embodiments, the population of aggregated cells includes at least one differentiated cell type. The differentiated cell type is selected based on the particular cellular makeup or characteristics of an injured, depleted or degenerated host tissue that is to be treated by a regenerative medical treatment method. For example, the differentiated cell type may include, but is not limited to, at least one differentiated cell type that is found in (1) a myocardium (e.g., cardiomyocytes, endothelial cells), (2) blood vessels (e.g., endothelial cells, smooth muscle cells), (3) pancreatic islets (e.g., α-cells, β-cells, δ-cells, γ-cells), (4) the liver (e.g., hepatocytes), (5) bone and cartilage (e.g., osteoblasts, osteoclasts, osteocytes, chondrocytes), (6) epidermis (e.g., epithelial cells, keratinocytes, melanocytes), (7) skeletal muscles and connective tissues (e.g., myocytes, fibroblasts, (8) a spinal cord (e.g., oligodendrocytes, motor neurons), (9) eyes (e.g., RPE cells), (10) one or more brain nuclei (e.g., dopaminergic neurons of the striatum and substantia nigra), (11) a hair follicle (e.g., dermal papilla cells), (12) a reproductive tissue (e.g., thecal cells, follicular cells, luteal cells, leydig cells, sertoli cells), (13) a kidney (e.g., glomerular parietal cells, podocytes, proximal tubule brush border cells), (14) bone marrow (e.g., hematopoietic stem cells (or parenchymal cells), mesenchymal stem cells (or marrow stromal cells), fibroblasts, plasma cells, stromal cells neutrophils, monocytes, myeloid cells, endothelial cells), (15) the gut (e.g., epithelial cells, parietal cells, gut endocrine cells such as L-cells).

As such, a population of aggregated cells that may be used in accordance with the embodiments described herein may be part of an MTP include, but is not limited to, a myocardial MTP that includes cardiomyocytes and/or endothelial cells; a vascular MTP that includes endothelial cells; smooth muscle cells; an islet MTP that includes α-cells, β-cells, δ-cells and/or γ-cells; a hepatic MTP that includes hepatocytes; an osteo MTP that includes osteoblasts, osteoclasts, osteocytes and/or chondrocytes, a dermal MTP that includes epithelial cells, keratinocytes, and/or melanocytes; a neuromuscular MTP that includes myocytes and/or fibroblasts; a motor MTP that includes oligodendrocytes and/or motor neurons; an ocular MTP that includes RPE cells; a dopiminergic MTP that includes dopaminergic neurons of the striatum and substantia nigra, a follicular MTP that includes dermal papilla cells; a female gonadal MTP that includes thecal cells, follicular cells, and/or luteal cells, a male gonadal MTP that includes leydig cells and/or sertoli cells; a renal MTP that includes glomerular parietal cells, podocytes, and/or proximal tubule brush border cells; marrow MTPs that include parenchymal cells, marrow stromal cells, fibroblasts, plasma cells, stromal cells neutrophils, monocytes, myeloid cells, and/or endothelial cells; and gut MTPs that includes epithelial cells or any segment of the gut, parietal cells, and/or gut endocrine cells. In certain embodiments, the MTP is a myocardial MTP which includes a population of aggregated cardiomyocytes.

Differentiated cell types that may be used in accordance with the embodiments described herein may be derived from a population of undifferentiated pluripotent, multipotent, or oligopotent stem cells or progenitor cells. In one embodiment, the undifferentiated cells are human cells. Examples of undifferentiated cells that may be used generate a differentiated cell type that is used in accordance with the embodiments described herein may include, but are not limited to, embryonic stem cells (ESC), embryonic germ cells (ESG), induced pluripotent stem cells (iPSC), adult stem cells, embryonic carcinoma cells (ECC), mesenchymal stem cells (MSC), circulating endothelial progenitor cells (EPCs), and bone marrow stem cells. In one embodiment, the undifferentiated cells are human ESCs (huESCs) or human iPSCs (huiPSCs). The one or more differentiated cell type or types differentiated target cells produced from the undifferentiated cells may be any suitable or desired differentiated target cell type including, but not limited to, cardiomyocytes, endothelial cells, smooth muscle cells, mesenchymal stem cells, pancreatic α-cells, pancreatic β-cells, pancreatic δ-cells, pancreatic γ-cells, osteoblasts, osteoclasts, osteocytes, chondrocytes, epithelial cells, keratinocytes, melanocytes, myocytes, fibroblasts, oligodendrocytes, motor neurons, RPE cells, dopaminergic neurons of the striatum and substantia nigra, hepatocytes, dermal papilla cells, thecal cells, follicular cells, luteal cells, leydig cells, sertoli cells glomerular parietal cells, podocytes, proximal tubule brush border cells, parenchymal cells, marrow stromal cells, fibroblasts, plasma cells, neutrophils, monocytes, myeloid cells, endothelial cells, gut epithelial cells, parietal cells, or gut endocrine cells.

In another embodiment, the differentiated cell types that may be used in accordance with the embodiments described herein may be derived from an established cell line or primary culture of cardiomyocytes, endothelial cells, smooth muscle cells, mesenchymal stem cells, α-cells, β-cells, δ-cells, γ-cells, osteoblasts, osteoclasts, osteocytes, chondrocytes, epithelial cells, keratinocytes, melanocytes, myocytes, fibroblasts, oligodendrocytes, motor neurons, RPE cells, dopaminergic neurons of the striatum and substantia nigra, hepatocytes, dermal papilla cells, thecal cells, follicular cells, luteal cells, leydig cells, sertoli cells glomerular parietal cells, podocytes, proximal tubule brush border cells, parenchymal cells, marrow stromal cells, fibroblasts, plasma cells, neutrophils, monocytes, myeloid cells, endothelial cells, gut epithelial cells, parietal cells, or gut endocrine cells. In some aspects, the differentiated cell types that may be used in accordance with the embodiments described herein may be derived from human endothelial cells, human cardiomyocytes, smooth muscle cells (e.g., aortic smooth muscle cells), mesenchymal stem cells, or a combination thereof.

According to some embodiments, the MTPs described herein may contain a single cell type (i.e., a “uni-cell MTP”). In other embodiments, the MTP is a bi-cell MTP which includes two cell types, a tri-cell MTP which includes three cell types, or a multi-cell MTP which includes four or more cell types.

In the case of bi-cell MTPs, tri-cell MTPs and multi-cell MTPs, the population of aggregated cells may include a first differentiated cell type; and one or more additional cell types, according to some embodiments. The one or more additional cell types may be a second differentiated cell type such as those described above, or may be any suitable pluripotent cell type, multipotent cell type, oligopotent cell type or a partially differentiated cell-type or terminally differentiated cell type. The one or more additional cell types may be a secondary or supportive cell which normally resides in a injured, depleted or degenerated tissue (i.e., a native tissue cell) or may be a cell that is able to divide and transform (or differentiate) into a secondary or supportive native tissue cell.

As described in the Example below, uni-cell MTPs, bi-cell MTPs, and tri-cell MTPs were made with varying cell composition that included hESC- or hiPSC-derived cardiomyocytes, human umbilical vein endothelial cells (HUVECs; Lonza), human mesenchymal stem cells (hMSCs; Lonza), human aortic smooth muscle cells (haSMCs; Lonza), or a combination thereof. Uni-cell MTPs that included hESC- or hiPSC-derived cardiomyocytes, HUVECs, or hMSCs were prepared; bi-cell vascular MTPs that included HUVECs and hMSCs or haSMCs were prepared, and tri-cell myocardial MTPs that included hESC- or hiPSC-derived cardiomyocytes, HUVECs, and hMSCs were prepared. For HUVEC-only MTPs, it was necessary to increase medium viscosity to facilitate cell aggregation overnight and this was done using 0.125% methyl cellulose. These MTPs may be referred to herein, alone or in combination, as cardiovascular MTPs according to some embodiments. Cardiovascular MTPs may include myocardial MTPs, vascular MTPs (aortic or venous), or a combination thereof.

In certain embodiments, methods of making or generating an MTP are provided herein. Such methods may be used to prepare/generate MTPs such as those described above, and include a step of culturing a population of cells that include at least one differentiated cell type in a minimally-adhesive culture system; and harvesting the MTPs. As referred to herein, a minimally-adhesive system includes a culture dish or plate to support the aggregation and/or association between individual cells of the population, but prevents or reduces attachment of the cells to the culture dish or plate, resulting in cell aggregates (i.e., MTPs) which maintain their cell-cell contact between each other. Suitable non-adhesive culture systems include, but are not limited to, a hanging-drop system, a microwell system, and a round-bottom plate system, all of which are described in the examples below.

As described above, the population of cells may include at least one differentiated cell type, and one or more additional cell-types may be included to produce bi-cell MTPs, tri-cell MTPs or multi-cell MTPs. The cell types which are included in the cultured population of cells may be seeded at any suitable amount or ratio. For example, a bi-cell MTP, which includes two cell types, cell-type 1 and cell-type 2, may be cultured using a ratio of 5:1, 4:1, 3:1, 2:1, 1:1, 1:2, 1:3, 1:4, 1:5 or any other suitable ratio of (cell-type 1):(cell-type 2). A tri-cell MTP, which includes three cell types, cell-type 1, cell-type 2, and cell-type 3, may be cultured using a ratio of 1:1:1, 2:1:1, 3:1:1, 4:1:1, 5:1:1, 1:2:1, 2:2:1, 3:2:1, 4:1:1, 5:2:1, 1:3:1, 2:3:1, 3:3:1, 4:3:1, 5:3:1, 1:4:1, 2:4:1, 3:4:1, 4:4:1, 5:4:1, 1:5:1, 2:5:1, 3:5:1, 4:5:1, 5:5:1, 1:1:2, 2:1:2, 3:1:2, 4:1:2, 5:1:2, 1:1:3, 2:1:3, 3:1:3, 4:1:3, 5:1:3, 1:1:4, 2:1:4, 3:1:4, 4:1:4, 5:1:4, 1:1:5, 2:1:5, 3:1:5, 4:1:5, 5:1:5, 1:2:2, 2:2:2, 3:2:2, 4:1:2, 5:2:2, 1:2:3, 2:2:3, 3:2:3, 4:1:3, 5:2:3, 1:2:4, 2:2:4, 3:2:4, 4:1:4, 5:2:4, 1:2:5, 2:2:5, 3:2:5, 4:1:5, 5:2:5, 1:3:2, 2:3:2, 3:3:2, 4:3:2, 5:3:2, 1:3:3, 2:3:3, 3:3:3, 4:3:3, 5:3:3, 1:3:4, 2:3:4, 3:3:4, 4:3:4, 5:3:4, 1:3:5, 2:3:5, 3:3:5, 4:3:5, 5:3:5, 1:4:2, 2:4:2, 3:4:2, 4:4:2, 5:4:2, 1:4:3, 2:4:3, 3:4:3, 4:4:3, 5:4:3, 1:4:4, 2:4:4, 3:4:4, 4:4:4, 5:4:4, 1:4:5, 2:4:5, 3:4:5, 4:4:5, 5:4:5, 1:5:2, 2:5:2, 3:5:2, 4:5:2, 5:5:2, 1:5:3, 2:5:3, 3:5:3, 4:5:3, 5:5:3, 1:5:4, 2:5:4, 3:5:4, 4:5:4, 5:5:4, 1:5:5, 2:5:5, 3:5:5, 4:5:5 or 5:5:5 or any other suitable ratio of (cell-type 1):(cell-type 2):(cell-type 3).

The culture system may also include a culture medium that includes one or more suitable components to optimize conditions for growing and/or maintaining a particular population of cells. For example, the media may include, but is not limited to, various concentrations of a basal medium (e.g., BME, DMEM, F-10, F-12, FMEM, IMDM, huEB, RPMI (e.g., RPMI-B27), EGM, EGM2 or any other classical or specialized commercial media available); an animal serum (e.g., fetal bovine serum (FBS)), one or more additional factors (e.g., methyl cellulose, growth factor, amino acids, vitamin); or a combination thereof. Further, one skilled in the art would understand that the culture medium is dictated by the type of cell, and a mixed population of cells may require a combination or mixture of several growth conditions to support the population's growth.

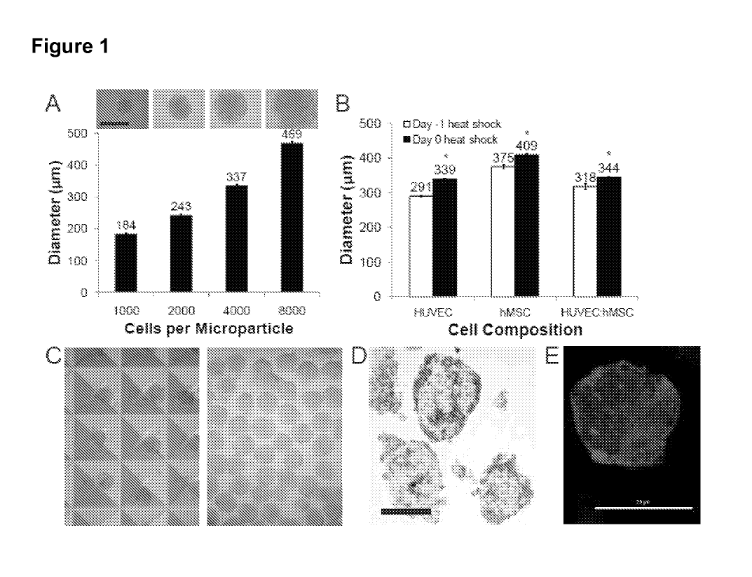

In some embodiments, the MTP may be generated to be of any suitable size for a non-invasive or minimally invasive delivery. To this end, the methods for producing the MTPs allow for scalable production, in that the size and diameter of an MTP is proportional to the number of cells seeded in each well or droplet of the culture system (see FIG. 1A). This precise control over cell composition and number allows for the generation of heterogeneous, spherical MTPs with a predictable diameter. In certain embodiments, the MTPs are generated to be less than approximately 1 mm in diameter, or less than approximately 500 μm. In other embodiments, the MTPs may be less than approximately 100 μm, less than approximately 200 μm, less than approximately 300 μm, less than approximately 400 μm, less than approximately 500 μm, less than approximately 600 μm, less than approximately 700 μm, less than approximately 800 μm, or less than approximately 900 μm. In other embodiments, the MTPs may be between approximately 1 and 100 μm, between approximately 100 and 200 μm, between approximately 200 and 300 μm, between approximately 300 and 400 μm, between approximately 400 and 500 μm, between approximately 500 and 600 μm, between approximately 600 and 700 μm, between approximately 700 and 800 μm, between approximately 800 and 900 μm, or between approximately 900 μm and 1 mm.

In some embodiments, the MTPs are administered by injection. As such, an MTP may be generated having a diameter that is smaller than the diameter of the needle used in accordance with these embodiments. Thus, if the desired needle used for administering the MTPs is a 22 gauge needle, the MTPs may be designed to have a diameter of less than approximately 400-420 μm. Due to the proportional relationship between the number of seeded cells and the resulting MTP diameter, an MTP having a diameter of less than approximately 400-420 μm is produced by seeding less than approximately 8000 cells per well. Differences in cell size will influence final MTP diameter (FIG. 1B). As such, the number of seeded cells may be adjusted based on the size of cells to be included in the MTP. For example, HUVECs are small cells relative to MSCs, therefore, an MTP which includes only HUVEC cells would require more cells per well to produce a desired MTP diameter than for an MTP which includes only MSC cells. Other needle sizes may be selected based on the target tissue, according to the standard of care.

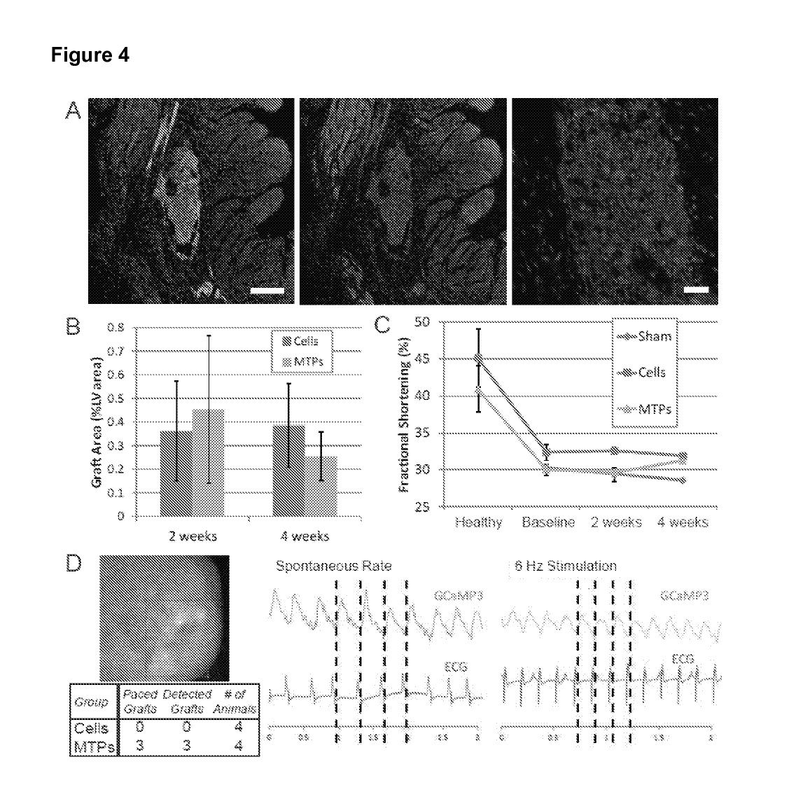

The MTPs described herein may be used for a broad range of cell-based therapies as described further below. In one embodiment, the MTPs described herein may be used in cell-based regenerative therapies is for the engraftment of cardiomyocytes in the heart after a myocardial infarction (heart attack). The placement of MTPs in the wall of the heart (intramyocardially) makes them comparable to injections of single cells (which is the current “gold standard” in cell transplantation for heart repair) in terms of their ease of delivery. Further, as described in Example 2 below, administration of MTPs facilitate the integration of the engrafted cells into the host organ in both structure and function (e.g., cellular alignment and electromechanical function). Upon assessing electrical integration of hESC-derived cardiomyocytes into infarcted rat hearts, it was demonstrated that MTPs were better able to couple to the host heart after 4 weeks (FIG. 4D) versus injection of a single cell suspension. The importance of electrical connectivity of graft with host is at least two-fold. First, hESC-derived cardiomyocytes that couple to the host are less likely to induce cardiac arrhythmias (Shiba et al. 2012). Second, electrical connectivity of the graft with the host is likely required prior to transplanted cells contributing to the mechanical function of the heart. Thus, the MTPs described herein are better suited for treatment and regenerating tissue because, when transplanted, more completely integrate with the host tissue functions.

In contrast to current systems, composition and methods, such as tissue patches, the micro-tissue particles can be injected directly to target organs, tissues, and/or other desired locations in a mammalian subject. Current tissue patches are not injectable and typically require implantation onto the surface of a target area which typically requires invasive surgical procedures and other unwanted complications.

The MTPs described herein offer at least the following advantages over the techniques currently available (e.g., macro-tissue patches, single cell suspension): (1) as compared to a single-cell suspension, the cells of an MTP maintain cell-cell and cell-matrix contacts during implantation, thereby improving cell survival; (2) MTP delivery to a host tissue is non-invasive or minimally invasive and can be accomplished, among other routes of administration, via a catheter and needle, whereas current tissue patches typically require implantation onto the surface of a target area which generally requires invasive surgical procedures and other unwanted complications; and (3) unlike many tissue patches, generation and implantation of MTPs does not require an engineered matrix scaffold, thereby reducing or eliminating adverse reactions by the host upon implantation.

### Pharmaceutical Compositions

According to some embodiments, the MTPs described herein may be part of a pharmaceutical composition. Such a pharmaceutical composition may include one or more MTP and a pharmaceutically acceptable carrier.

A “pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. Such a carrier may comprise, for example, a liquid, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, or some combination thereof.

Each component of the carrier is “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) natural polymers such as gelatin, collagen, fibrin, fibrinogen, laminin, decorin, hyaluronan, alginate and chitosan; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as trimethylene carbonate, ethyl oleate and ethyl laurate; (13) agar; (14) buffering agents, such as magnesium hydroxide and aluminum hydroxide; (15) alginic acid (or alginate); (16) pyrogen-free water; (17) isotonic saline; (18) Ringer's solution; (19) alcohol, such as ethyl alcohol and propane alcohol; (20) phosphate buffer solutions; (21) thermoplastics, such as polylactic acid, polyglycolic acid, (22) polyesters, such as polycaprolactone; (23) self-assembling peptides; and (24) other non-toxic compatible substances employed in pharmaceutical formulations such as acetone.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

In one embodiment, the pharmaceutically acceptable carrier is an aqueous carrier, e.g. buffered saline and the like. In certain embodiments, the pharmaceutically acceptable carrier is a polar solvent, e.g. acetone and alcohol.

The concentration of MTPs in these formulations can vary widely, and will be selected primarily based on fluid volumes, viscosities, organ size, body weight and the like in accordance with the particular mode of administration selected and the biological system's needs.

The pharmaceutical composition may include a single MTP type or a combination of more than one MTP types. For example, the pharmaceutical composition may include a uni-cell myocardial MTP produced from cardiomyocytes, a bi-cell vascular MTP produced from endothelial cells and MSCs, a tri-cell myocardial MTP produced from cardiomyocyte, endothelial cells and MSCs; or a combination thereof.

In some embodiments, the pharmaceutical compositions may also include one or more graft-enhancing agents to increase the efficacy of integration with the host target tissue. Graft-enhancing agents that may be used in accordance with the embodiments described herein include, but are not limited to, immunosuppressive agents (e.g., cyclosporine A), antibiotics, extracellular matrix elements (e.g., Matrigel®, collagen, gelatin, fibronectin, fibrinogen, fibrin, laminin), anti-apoptotic agents, anti-ischemic agents, growth or differentiation factors, pro-proliferation agents, and anti-toxicity agents.

In one embodiment, the one or more graft enhancing agents may include a pro-survival cocktail, which includes a plurality of pro-survival agents. The plurality of pro-survival agents may include, but are not limited to, two or more of the following: Matrigel, a cell-permeant peptide from Bcl-XL, cyclosporine A, a compound that opens ATP-dependent potassium channels (e.g., pinacidl), IGF-1, and a caspase inhibitor (e.g., ZVAD-fmk). In certain aspects, the pro-survival cocktail may have a formulation such as that found in U.S. Pat. No. 7,875,451 to Murry and Laflamme, which is hereby incorporated by reference as if fully set forth herein,

### Therapeutic Uses of Micro-Tissue Particles and Pharmaceutical Compositions Thereof

According to the embodiments described herein, an MTP or a pharmaceutical composition thereof may be used to treat a target tissue that has been injured, has degraded, or is lacking in a subject. As such, methods for treating, repairing or replacing a target tissue are provided. Such methods may include a step of administering a therapeutically effective amount of one or more MTPs (such as those described above) or a pharmaceutical composition thereof.

Target tissues that may be treated in accordance with the methods described herein may include, but are not limited to, myocardial tissue, blood vessels (arteries such as coronary arteries or aorta, or veins), a pancreas (e.g., pancreatic islets), bone, cartilage, skeletal muscle, tendons, ligaments, epidermis, spinal cord, eyes, nervous tissue (e.g., brain nuclei, motor neurons, nerves), liver, hair follicle, ovary, testis, kidney, bone marrow, and gut (e.g., intestines, stomach). Such target tissues may need treatment as a result of an acute or pathological injury or condition including, but not limited to, myocardial infarction, heart failure, atherosclerosis, angioplasty, limb ischemia, diabetes, multiple sclerosis, Parkinson's disease, Huntington's disease, spinal cord injury, musculoskeletal injury (e.g., bone fractures, tendon or cartilage tears), arthritis, osteoporosis, cuts or gashes, and ocular injuries, degenerative diseases (e.g., macular dystrophy, macular degeneration, glaucoma), baldness, cirrhosis of the liver, liver damage from Hepatitis or drug/toxin exposure, infertility, bone marrow transplantation after chemotherapy, kidney failure, Crohn's disease, or ulcerative colitis, In some embodiments, the method includes administration of MTPs for neo-vascular therapy and/or cardiac regeneration therapy after injury to the myocardium (e.g., after a myocardial infarction or prolonged ischaemic event) or the vasculature. For example, the method may include a regenerative treatment for a denuded or injured arterial wall following a coronary angioplasty procedure or following repair of an aneurysm, wherein a patient is administered vascular MTPs (e.g., aortic or arterial vascular MTPs that include aortic or arterial smooth muscle cells, endothelial cells, MSCs, or a combination thereof). In another embodiment the method includes a regenerative treatment for myocardial infarction, wherein a patient is administered myocardial MTPs in an amount effective to treat myocardial infarction, thereby restoring electromechanical function of the myocardial tissue.

The terms “treat,” “treating,” or “treatment” as used herein with regards to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. For example, a treatment with an MTP or a pharmaceutical composition thereof may refer to replacement of an injured, degenerated or absent tissue, which results in an improved tissue function, thereby preventing a condition associated with the target tissue, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. The treatments described herein may be used in any suitable subject, including a human subject or any mammalian or avian subject that needs treatment in accordance with the methods described herein (e.g., dogs, cats, horses, rabbits, mice, rats, pigs, cows).

In some embodiments, the method of treatment may include administering at least one graft-enhancing agent in combination with the pharmaceutical composition. “In combination” or “in combination with,” as used herein, means in the course of treating the same target tissue, disease or condition in the same patient using two or more agents, drugs, treatment regimens, treatment modalities or a combination thereof, in any order. This includes simultaneous administration, as well as in a temporally spaced order of up to several days apart. Such combination treatment may also include more than a single administration of any one or more of the agents, drugs, treatment regimens or treatment modalities. Further, the administration of the two or more agents, drugs, treatment regimens, treatment modalities or a combination thereof may be by the same or different routes of administration.

Graft-enhancing agents that may be used in accordance with the embodiments described herein include, but are not limited to, immunosuppressive agents (e.g., cyclosporine A), antibiotics, extracellular matrix elements (e.g., Matrigel®, collagen, gelatin, fibronectin, fibrinogen, fibrin, laminin), anti-apoptotic agents, anti-ischemic agents, growth or differentiation factors, pro-proliferation agents, and anti-toxicity agents. In one embodiment, the at least one graft enhancing agent may be a pro-survival cocktail, which includes a plurality of pro-survival agents. The plurality of pro-survival agents may include, but are not limited to, two or more of the following: Matrigel, a cell-permeant peptide from Bcl-XL, cyclosporine A, a compound that opens ATP-dependent potassium channels (e.g., pinacidl), IGF-1, and a caspase inhibitor (e.g., ZVAD-fmk). In certain aspects, the pro-survival cocktail may have a formulation such as that found in U.S. Pat. No. 7,875,451 to Murry and Laflamme, which is hereby incorporated by reference as if fully set forth herein,

An MTP or a pharmaceutical composition thereof can be administered to a biological system by any administration route known in the art, including without limitation, oral, enteral, buccal, nasal, topical, rectal, vaginal, aerosol, transmucosal, epidermal, transdermal, dermal, ophthalmic, pulmonary, subcutaneous, and/or parenteral administration. The pharmaceutical compositions can be administered in a variety of unit dosage forms depending upon the method of administration. In one embodiment, the MTPs or a pharmaceutical composition thereof is administered parenterally.

A parenteral administration refers to an administration route that typically relates to injection which includes but is not limited to intravenous, intramuscular, intraarterial, intrathecal, intracapsular, intraorbital, intra cardiac, intradermal, intraperitoneal, transtracheal, subcutaneous, subcuticular, intraarticular, subcapsular, subarachnoid, intraspinal, and/or intrasternal injection and/or infusion. The injection may be administered directly through the skin as directed by a clinician, or in some embodiments, may be administered by way of catheterization via the femoral artery or any other suitable vessel or lumen. For example, in the case of a myocardial MTP for treatment of a myocardial infarction, the MTP or composition thereof may be injected directly into the heart by way of catheterization. In some embodiments, the injection may be administered using a needle having a gauge size suitable for the target tissue. Suitable needle gauge sizes may include, but are not limited to, an 18 gauge needle, a 19 gauge needle, a 20 gauge needle, a 21 gauge needle, a 22 gauge needle, a 23 gauge needle, a 24 gauge needle, a 25 gauge needle, a 26 gauge needle, a 27 gauge needle, a 28 gauge needle, a 29 gauge needle, or a 30 gauge needle.

An MTP or a pharmaceutical composition thereof can be given to a subject in the form of formulations or preparations suitable for each administration route. The formulations useful in the methods of the invention include one or more MTPs, one or more pharmaceutically acceptable carriers therefor, and optionally other therapeutic ingredients. The formulations may conveniently be presented in unit dosage form and may be prepared by any methods well known in the art of pharmacy. The amount of active ingredient which can be combined with a carrier material to produce a single dosage form will vary depending upon the subject being treated and the particular mode of administration. The amount of an MTP, which can be combined with a carrier material to produce a pharmaceutically effective dose, will generally be that amount of an MTP which produces a therapeutic effect.

Methods of preparing these formulations or compositions include the step of bringing into association an MTP with one or more pharmaceutically acceptable carriers and, optionally, one or more accessory ingredients. In general, the formulations are prepared by uniformly and intimately bringing into association an MTP with liquid carriers, or finely divided solid carriers, or both.

Formulations suitable for parenteral administration comprise an MTP in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e.g., such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents, viscous agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In an embodiment of the invention, an MTP or composition thereof is delivered to a disease or infection site in a therapeutically effective dose. A “therapeutically effective amount” or a “therapeutically effective dose” is an amount of an MTP that produces a desired therapeutic effect in a subject, such as preventing or treating a target condition or alleviating symptoms associated with the condition. The most effective results in terms of efficacy of treatment in a given subject will vary depending upon a variety of factors, including but not limited to the characteristics of the MTP, the size and fragility of the injured tissue, the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005.

In some embodiments, a therapeutically effective amount is at least approximately 5000 MTPs (e.g., at least 5 million cells total), or at least approximately 10,000 MTPs, but may be more or less, depending on the size of the injured target tissue. Thus, in some embodiments, a therapeutically effective amount is less than approximately 5000 MTPs, approximately 5,000 MTPs, greater than approximately 5000 MTPs, approximately 10,000 MTPs, or greater than approximately 10,000 MTPs. The pharmaceutically effective dose may be delivered in a single dose, or may be divided into 2 or more partial doses. For example, a pharmaceutically effective dose of 5000 MTPs, each MTP including 1000 cells for a total of 5 million cells may be administered in 2 injections of 2500 MTPs, 10 injections of 500 MTPs, or any other suitable number of partial doses. In some embodiments, the effective dose is sufficient for administration by injection into the body for regenerative therapy. In other embodiments, the effective dose is sufficient for administration by injection into the body for use in treating damaged heart.

The following examples are provided to better illustrate the various embodiments of the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. For example, although the examples below describe MTP generation and use in the complex setting of heart repair for myocardial infarction as both a neo-vascular therapy and a cardiac regeneration therapy where human embryonic stem cells (hESCs) and human induced pluripotent stem cells (hiPSCs) are used to derive human cardiomyocytes, one skilled in the art would understand that MTPs with a different cellular make-up may also be generated for use in other clinical conditions or outcomes. Further, all references cited in the disclosure are hereby incorporated by reference in their entirety, as if fully set forth herein.

### EXAMPLES

### Example 1

### Formation of Micro-Tissue Particles (MTPs)

### Materials and Methods

Maintenance of Undifferentiated Pluripotent Stem Cells. Undifferentiated H7 hESCs (WA07; WiCell Research Institute) were cultured in feeder-free conditions as previously described¹ using MEF-conditioned medium and tissue culture plates coated with Matrigel (BD Biosciences). Undifferentiated IMR90 human iPSCs were maintained on irradiated MEFs and transitioned to feeder-free culture conditions for 2-3 passages prior to directed differentiation.

Cardiac Directed Differentiation. Human cardiomyocytes were derived from hESCs and hiPSCs using the high-density monolayer differentiation described by Laflamme et al (Laflamme et al. 2007). Briefly, undifferentiated pluripotent stem cells were dispersed into single cells, replated in 6-well or 24-well plates, and allowed to grow to super-confluency in MEF-conditioned medium. Activin A (100 ng mL⁻¹) was applied for 18-24 hr followed by BMP4 (10 ng mL⁻¹) for 4 days in RPMI medium (Gibco) with B27 supplement without insulin (Invitrogen). Medium was changed on day 5 of differentiation and every 2-3 days until the cells were used. RPMI medium (Gibco) with B27 supplement (containing insulin; Invitrogen) was used to feed cells after day 7.

Formation of Micro-Tissue Particles (MTPs). Initial experiments used hanging drops or round-bottom 96-well plates to form micro-tissue particles. Hanging drops were formed with 25 μL/drop using a multichannel pipette on the lid of a 150 mm circular plate (Corning). The lid was carefully inverted and 10 mL PBS was added to the bottom of the plate to maintain humidity. In 96-well round-bottom plates, 25 μL per well was used and MTPs made from endothelial cells alone were easily dislodged by tapping the plate after overnight incubation at 37° C.

To increase throughput (particularly for implantation studies), MTPs were formed in microwells using AggreWell™ 400 plates (STEMCELL Technologies) with approximately 1200 microwells per well of a 24-well plate, at between 1000 and 8000 cells per microwell. MTPs were cultured overnight (18-20 hrs) or up to 5 days in microwells with a partial change of culture medium (1.0-1.5 mL of 2.0 mL total per well) every other day.

MTPs were made with varying cell composition, including one, two, or three cell types, namely hESC- or hiPSC-derived cardiomyocytes, human umbilical vein endothelial cells (HUVECs; Lonza), or human mesenchymal stem cells (hMSCs; Lonza). Bi-cell “vascular” MTPs contained HUVECs plus hMSCs, and tri-cell “myocardial” MTPs contained hESC- or hiPSC-derived cardiomyocytes, HUVECs, and hMSCs. For HUVEC-only MTPs, it was necessary to increase medium viscosity to facilitate cell aggregation overnight and this was done using 0.125% methyl cellulose.

Medium conditions were optimized for each cell composition and therefore were varied depending on cell type. Media conditions tested were: huEB, RPMI-B27, RPMI-B27 with 20% FBS, and RPMI-B27 with 0.125% methyl cellulose for cardiomyocyte-only MTPs; EGM2 and EGM2 with 0.125% methyl cellulose for HUVEC-only MTPs; and 50/50 (v/v) huEB/EGM2 for “vascular” and “myocardial” MTPs or 1:1:1 huEB: EGM2: RPMI-B27 for “myocardial” MTPs.

Harvesting MTPs. MTPs were harvested with different techniques based on the format by which they were formed. These methods were most labor-intensive for hanging drops and 96-well plates. For hanging drops, MTPs were washed off the lid and into the bottom of the 150 mm plate with PBS. MTPs were harvested from 96-well round bottom plates by tapping the plate on the bench top to dislodge the MTPs and then collected in a 150 mm plate containing PBS using a multichannel pipette. MTPs were harvested from microwells by gently pipetting medium around the well to wash MTPs out of microwells, as cells do not readily adhere to the PDMS substrate. MTPs were collected in a 50 mL conical and either allowed to settle by gravity (20 min) or centrifuged into a pellet (1000 rpm, 3 min) to remove medium and PBS then resuspended in PBS to wash away residual medium, centrifuged again and resuspended in the desired solution for implantation or fixation.

Immunohistochemistry. MTPs were fixed in 4% paraformaldehyde for 30 min, rinsed with PBS, transferred to a 1.7 mL eppendorf tube, spun into a pellet with a tabletop centrifuge, and then embedded as a pellet in HistoGel (Thermo Scientific) according to manufacturer instructions. Briefly, the pellet of MTPs was resuspended in 250 μL of warmed HistoGel, immediately spun into a pellet with a tabletop centrifuge, and then put on ice until the HistoGel was firm. A needle was inserted along the side of the tube and PBS was injected to dislodge the gel pellet from the eppendorf. Excess gel was trimmed away from the pellet, which was then wrapped in lens paper and put into a cassette for routine processing, paraffin embedding, and sectioning (4 μm thick).

Analysis. MTP diameter was measured from images (50-200× magnification) of MTPs collected into a well or plate prior to fixation using ImageJ. Statistical significance (p<0.05) was determined using a two-tailed Student's t test assuming unequal variance. Error bars represent SEM for all measurements.

### Results

Micro-tissue particles (MTPs) were uniform in size and spherical in shape using any of three methods (microwells, hanging drops, or 96-well round-bottom plate) for overnight formation. Varying the input cell number enabled precise control of MTP diameter (FIG. 1A). Because cell size also varies by cell type, cell composition influenced MTP diameter (FIG. 1B). Further, when cells are exposed to heat shock (42° C., 30 min)—a procedure that promotes cell survival upon implantation—MTPs successfully form, but have slightly altered diameter (FIG. 1B). The greatest throughput of MTP formation was achieved in microwells (using Aggrewell400™ plates from STEMCELL Technologies; FIG. 1C). When HUVECs and hMSCs are mixed in “vascular” MTPs or together with cardiomyocytes in “myocardial” MTPs, the endothelial cells create a desirable network of interconnected cells and do not segregate from the other cell populations (FIG. 1D).

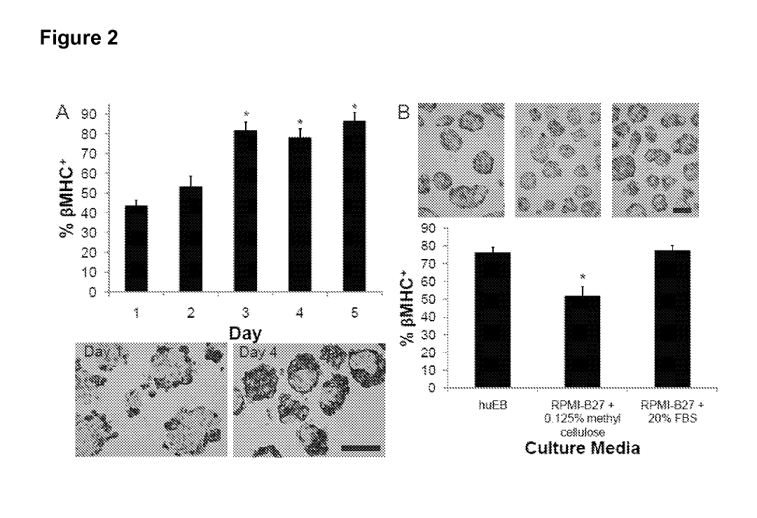

Optimization of medium conditions demonstrated that matching culture medium to cell type was crucial for maintaining cell viability and forming MTPs. Cardiac-only MTPs were cultured for up to 5 days and showed enrichment for cardiomyocytes up to ˜80% βMHC⁺ cells by day 3 from a ˜40% cardiomyocyte input population (FIG. 2A), as previously observed for macroscopic scaffold-free cardiac tissue patches.¹ Further, cardiac-only MTPs maintained robust cardiomyocytes when grown in different media that all support cardiomyocyte growth, including huEB, ½ huEB+½ EGM2, RPMI-B27, or RPMI-B27 with 20% FBS (FIG. 2 A, B). RPMI-B27 with 0.125% methyl cellulose showed a lower percent cardiomyocytes after 4 days, suggesting that methyl cellulose hindered cardiac enrichment (FIG. 2B). However, addition of 0.125% methyl cellulose to EGM2 increased the viscosity of the medium and was necessary to form HUVEC-only MTPs (FIG. 1A), which may be due to the preference of endothelial cells to form sheets (e.g. to line lumens) rather than form cellular aggregates. Vascular MTPs had robust HUVECs and hMSCs when formed in a mixed medium of 50% EGM2 and 50% huEB (FIG. 1C).

Micro-tissue particles (MTPs) provide a dense, cellular engineered tissue that is amenable to minimally invasive transplantation via catheter and needle to any region of the body. As described herein, a number of methods for forming MTPs have been demonstrated, including methods which utilize microwells for high throughput, and have used different combinations of cells for creating uni-, bi-, and tri-cellular MTPs. In addition, vascularized MTPs were made, which resulted in more rapid angiogenesis and vessel formation after transplantation. Further, any cell type can be combined with vascular cells for a multicellular implant to reflect the cell populations of the host tissue.

### Example 2

### Model of Myocardial Infarction and Implantation of MTPs

### Materials and Methods

Model of Myocardial Infarction and Implantation of MTPs. All animal procedures were conducted in accordance with U.S. National Institutes of Health Policy on Humane Care and Use of Laboratory Animals and approved by the University of Washington (UW) Animal Care Committee. Rats were housed in the Department of Comparative Medicine and cared for in accordance with UW Institutional Animal Care and Use Committee (IACUC) procedures. Male athymic Sprague Dawley rats (250 g) were anesthetized with isofluorane, intubated, and mechanically ventilated. A thoracotomy exposed the heart and the pericardium was partially removed. To enhance cell survival, MTPs were treated with heat shock at 42° C. for 40 min one day prior to implantation and prepared in a pro-survival cocktail as previously described (Laflamme 2007; U.S. Pat. No. 7,875,451). MTPs were harvested from microwells, rinsed in PBS, and suspended in 50/50 DMEM/growth-factor-reduced Matrigel™ (BD Biosciences) with pro-survival cocktail in a total of approximately 90 μL.

Three injections were made of about 30 μL each using a 24 g needle on a 100 μL Hamilton syringe and were located in the center of the infarct and in the lateral and medial border zones. Prior to injection, 8.0 suture was used to create a purse-string suture, which was closed immediately after retraction of the needle. Little or no leakage of MTPs out of the injection sites was observed. The chest was closed aseptically and animal recovery was monitored. To prevent cell death via mitochondrial pathways, animals received cyclosporine A (0.75 mg/day; Wako Pure Chemicals) subcutaneously for one week beginning one day prior to implantation.

Animals were sacrificed 1 or 2 week(s) after MTP implantation and hearts removed. Whole hearts were thoroughly rinsed in PBS, fixed in 4% paraformaldehyde overnight, cut into 2 mm sections, processed, and paraffin-embedded for sectioning and histology.

Effectiveness of the engraftment was realized with injecting at least 5000 micro-tissue particles, each particle having approximately 1000 cells for a total of 5 million cells. Additionally, effective engraftment was realized with 10,000 micro-tissue particles. Volumes and numbers of micro-tissue particles higher than at least about 5,000 can be effectively used and the size of the needle can be varied to accommodate varying micro-tissue particle volumes and numbers. Similarly, sizes and numbers of cells per micro-tissue particle higher than at least 1,000 cells per micro-tissue particle can be effectively used for engraftment with the size of the needle being varied to accommodate various sizes and numbers of cells per cell particle.

Echocardiography was used to assess heart function with a GE Vivid 7 echocardiography system. Fractional shortening (%) was assessed as (LVEDD-LVESD)/LVESD. Animals were sacrificed 1, 2, or 4 week(s) after MTP implantation and hearts removed for live ex vivo GCaMP3 fluorescence imaging (Stevens et al. 2009) and subsequent fixation. Whole hearts were thoroughly rinsed in PBS, fixed in 4% paraformaldehyde overnight, cut into 2 mm sections, processed, and paraffin-embedded for sectioning and histology.

Immunohistochemistry. Rat hearts were rinsed in PBS three times and fixed in 4% paraformaldehyde overnight at 4° C. Hearts were sliced at 2 mm thickness and put into cassettes for routine processing, paraffin embedding, and sectioning.

Picrosirius red stain with fast green counterstain was used to determine infarct area. Immunohistochemistry for beta-myosin heavy chain (βMHC; clone A4.951, American Type Culture Collection), human CD31 (hCD31; Dako), and cardiac troponin T (cTnT; 1:100; NeoMarkers) is as previously published (Stevens et al. 2009; Laflamme et al. 2007; Kreutziger et al. 2011) for MTPs and rat heart sections with an additional antigen retrieval for βMHC and cTnT as follows. After deparafinization and rehydration, slides were boiled in 0.01 M citrate buffer (1.8 mM citric acid, 8.2 mM sodium citrate) for 10 min then allowed to cool for 20 min in the buffer and washed in PBS (5 minutes) before routine blocking in 1.5% normal goat serum, overnight incubation in primary antibody, and chromagenic detection by diaminobenzidine (DAB; Sigma) and hematoxylin counterstain. To label human cells, in situ hybridization was done with a human pan-centromeric genomic probe with detection by DAB and detection of preceding immunohistochemistry with Vector Red (Vector Laboratories).

### Results

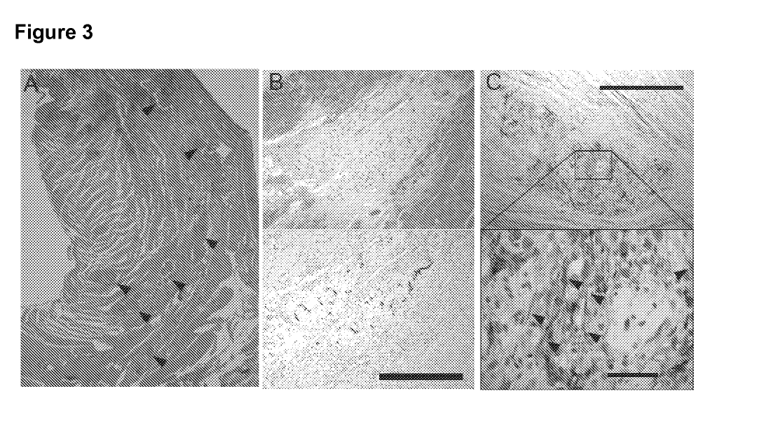

MTPs were implanted by needle injection in athymic rat hearts and showed engraftment at 1 week. Myocardial infarction was induced by ischemia/reperfusion and MTPs were implanted in the acute granulation phase of injury 4 days later during a second surgery. To show MTP distribution in the heart wall immediately after injection, one animal was sacrificed after injection (FIG. 3A). Engrafted MTPs were found dispersed through the left ventricular (LV) wall and made up 3% of the LV area, while infarct scar was 17% of LV area. Tri-cell MTPs had cardiomyocytes, HUVECs and hMSCs (2:2:1) and created human grafts in the host heart tissue with hCD31⁺ vessel-like structures at one week (FIG. 3B). Further, robust cardiomyocyte staining with the development of striations characteristic of cardiac muscle demonstrated cardiomyocyte engraftment (FIG. 3C).

To assess the efficacy of MTP implantation versus the current “gold” standard in treatment, grafts of MTPs were compared to grafts of single cardiomyocytes at 2 and 4 weeks. Intramyocardial grafts were GFP-positive (green, indicating the presence of GCaMP3 in the implanted cells) and engrafted cardiomyocytes were double-labeled for α-actinin (red) and showed sarcomeric development (FIG. 4A). By histology, MTP grafts were equivalent in size to single cardiomyocyte “Cell” grafts (FIG. 4B). Assessment of heart function by echocardiography measurements of fractional shortening showed no difference in the MTP and single Cell treatment groups (FIG. 4C), suggesting that MTPs are equivalent to current standards for cell transplantation in a myocardial infarction model. Interestingly, when the electrical coupling of the graft to the host was assessed by ex vivo imaging of the transplanted, GCaMP3-positive cardiomyocytes, the MTP grafts proved to be superior (FIG. 4D). The coupling of the graft to host was present in 3 of 4 hearts in the MTP group under both spontaneous and stimulated excitation up to 6 Hz. In stark contrast, zero single Cell-derived grafts were detected by ex vivo imaging (FIG. 4D, table).

In summary, MTPs provide a novel avenue to cell transplantation of engineered tissue that is less invasive than macroscopic engineered tissues, maintains cell-cell and cell-matrix interactions and geometry during implantation, is simple to produce, and can be customized for many applications.

The Examples above have demonstrated the formation, implantation, and engraftment of a novel type of engineered tissue for cardiac repair. These micro-tissue particles overcome a number of challenges in the field of cell-based cardiac therapies by creating a scaffold-free, micron-sized tissue that is deliverable via needle into the wall of the heart. Different cellular formulations—including cardiac, vascular, and myocardial micro-tissue particles—engraft in the heart and create new tissue, regenerating that lost to ischemic injury (heart attack).

### REFERENCES

The references, patents and published patent applications listed below, and all references cited in the specification above are hereby incorporated by reference in their entirety, as if fully set forth herein.
Kreutziger, K. L., et al. Developing vasculature and stroma in engineered human myocardium. Tissue Eng Part A 17, 1219-1228 (2011).
Laflamme, M. A., et al. Cardiomyocytes derived from human embryonic stem cells in pro-survival factors enhance function of infarcted rat hearts. Nat Biotechnol 25, 1015-1024 (2007).
Shiba, Y., et al. Human ES-cell-derived cardiomyocytes electrically couple and suppress arrhythmias in injured hearts. Nature 489, 322-325 (2012).
Stevens, K. R., et al. Physiological function and transplantation of scaffold-free and vascularized human cardiac muscle tissue. Proc Natl Acad Sci USA 106, 16568-16573 (2009).

What is claimed is:

1. A micro-tissue particle comprising a scaffold-free population of aggregated cells having a diameter less than approximately 1 mm, the population comprising at least one terminally differentiated cell type.

2. The micro-tissue particle of claim 1, wherein the diameter is less than approximately 500 μm

3. The micro-tissue particle of claim 1, wherein the at least one terminally differentiated cell type is selected from cardiomyocytes, endothelial cells, smooth muscle cells, pancreatic α-cells, pancreatic β-cells, pancreatic δ-cells, pancreatic γ-cells, osteoblasts, osteoclasts, osteocytes, chondrocytes, epithelial cells, keratinocytes, melanocytes, myocytes, fibroblasts, oligodendrocytes, motor neurons, RPE cells, dopaminergic neurons, hepatocytes, dermal papilla cells, thecal cells, follicular cells, luteal cells, leydig cells, sertoli cells glomerular parietal cells, podocytes, proximal tubule brush border cells, parenchymal cells, marrow stromal cells, fibroblasts, plasma cells, neutrophils, monocytes, myeloid cells, endothelial cells, gut epithelial cells, parietal cells, or gut endocrine cells or a combination thereof.

4. The micro-tissue particle of claim 1, wherein the at least one terminally differentiated cell type is an endothelial cell and a cardiomyocyte.

5. The micro-tissue particle of claim 1, wherein the at least one terminally differentiated cell type is derived from a human embryonic stem cell (hESC) or an induced pluripotent stem cell.

6. The micro-tissue particle of claim 1, further comprising a mesenchymal stem cell.

7. A pharmaceutical composition comprising a micro-tissue particle and a carrier, the micro-tissue particle comprising a scaffold-free population of aggregated cells having a diameter less than approximately 1 mm, the population comprising at least one terminally differentiated cell type.

8. The pharmaceutical composition of claim 7, wherein the diameter is less than approximately 500 μm

9. The pharmaceutical composition of claim 7, wherein the at least one terminally differentiated cell type is selected from cardiomyocytes, endothelial cells, smooth muscle cells, pancreatic α-cells, pancreatic β-cells, pancreatic δ-cells, pancreatic γ-cells, osteoblasts, osteoclasts, osteocytes, chondrocytes, epithelial cells, keratinocytes, melanocytes, myocytes, fibroblasts, oligodendrocytes, motor neurons, RPE cells, dopaminergic neurons, hepatocytes, dermal papilla cells, thecal cells, follicular cells, luteal cells, leydig cells, sertoli cells glomerular parietal cells, podocytes, proximal tubule brush border cells, parenchymal cells, marrow stromal cells, fibroblasts, plasma cells, neutrophils, monocytes, myeloid cells, endothelial cells, gut epithelial cells, parietal cells, gut endocrine cells or a combination thereof.

10. The pharmaceutical composition of claim 7, wherein the at least one terminally differentiated cell type is an endothelial cell and a cardiomyocyte.

11. The pharmaceutical composition of claim 7, wherein the at least one terminally differentiated cell type is derived from a human embryonic stem cell (hESC) or an induced pluripotent stem cell.

12. The pharmaceutical composition of claim 7, further comprising a mesenchymal stem cell.

13. The pharmaceutical composition of claim 7, further comprising one or more graft-enhancing agents selected from immunosuppressive, antibiotics, extracellular matrix elements, anti-apoptotic agents, anti-ischemic agents, anti-toxicity agents, growth or differentiation factors, pro-proliferation agents, pro-survival agents, or a combination thereof.

14. The pharmaceutical composition of claim 13, wherein the one or more graft-enhancing agents is cyclosporine A.

15. A method for treating an acute or pathologically injured target tissue comprising administering a therapeutically effective amount of a pharmaceutical composition, the pharmaceutical composition comprising a micro-tissue particle comprising a scaffold-free population of aggregated cells having a diameter less than approximately 1 mm, the population comprising at least one terminally differentiated cell type.

16. The method of claim 15, wherein the diameter is less than approximately 500 μm.

17. The method of claim 15, wherein the at least one terminally differentiated cell type is selected from cardiomyocytes, endothelial cells, smooth muscle cells, pancreatic α-cells, pancreatic β-cells, pancreatic δ-cells, pancreatic γ-cells, osteoblasts, osteoclasts, osteocytes, chondrocytes, epithelial cells, keratinocytes, melanocytes, myocytes, fibroblasts, oligodendrocytes, motor neurons, RPE cells, dopaminergic neurons, hepatocytes, dermal papilla cells, thecal cells, follicular cells, luteal cells, leydig cells, sertoli cells glomerular parietal cells, podocytes, proximal tubule brush border cells, parenchymal cells, marrow stromal cells, fibroblasts, plasma cells, neutrophils, monocytes, myeloid cells, endothelial cells, gut epithelial cells, parietal cells, gut endocrine cells or a combination thereof.

18. The method of claim 15, wherein the at least one terminally differentiated cell type is an endothelial cell and a cardiomyocyte.

19. The method of claim 15, wherein the at least one terminally differentiated cell type is derived from a human embryonic stem cell (hESC) or an induced pluripotent stem cell.

20. The method of claim 15, further comprising a mesenchymal stem cell.

21. The method of claim 15, wherein the pharmaceutical composition further comprising one or more graft-enhancing agents selected from immunosuppressive, antibiotics, extracellular matrix elements, anti-apoptotic agents, anti-ischemic agents, anti-toxicity agents, growth or differentiation factors, pro-proliferation agents, pro-survival agents, or a combination thereof.

22. The method of claim 15, wherein the pharmaceutical composition is administered by injection.

23. The method of claim 15, wherein the therapeutically effective dose comprises at least 5000 MTPs.

24. The method of claim 15, wherein the acute or pathologically injured target tissue is a myocardial tissue, a blood vessel, a pancreatic islet, a bone, cartilage, a skeletal muscle, a tendon, a ligament, an epidermis, a spinal cord, an eye, a nervous tissue, a liver, a hair follicle, an ovary, a testis, a kidney, bone marrow, an intestine, or a stomach.

25. The method of claim 24, wherein the target tissue is injured, degenerated or missing due to a myocardial infarction, heart failure, atherosclerosis, an angioplasty treatment, limb ischemia, diabetes, multiple sclerosis, Parkinson's disease, Huntington's disease, a spinal cord injury, a musculoskeletal injury, a bone fractures, a muscle tear, a tendon or cartilage tear, arthritis, osteoporosis, a cuts or gash, macular dystrophy, macular degeneration, glaucoma, baldness, cirrhosis of the liver, liver damage from Hepatitis or drug/toxin exposure, infertility, bone marrow transplantation after chemotherapy, kidney failure, Crohn's disease, or ulcerative colitis.

26. The method of claim 15, further comprising administering one or more graft-enhancement agents in combination with the pharmaceutical composition, wherein the one or more graft-enhancement agents are selected from immunosuppressive, antibiotics, extracellular matrix elements, anti-apoptotic agents, anti-ischemic agents, anti-toxicity agents, growth or differentiation factors, pro-proliferation agents, pro-survival agents, or a combination thereof.
